(19) United States
(12) Patent Application Publication
(10) Pub. No.: US 2024/0306604 A1
(43) Pub. Date: Sep. 19, 2024

(54) SYSTEMS AND METHODS FOR BRAIN-MACHINE-INTERFACE-AIDED FEDERATED TRAINING OF SCENT DETECTION ANIMALS

(71) Applicants: New York University, New York, NY (US); Canaery Inc., Media, PA (US)

(72) Inventors: Dmitry RINBERG, New York, NY (US); Gabriel LAVELLA, Media, PA (US); Peter LEDOCHOWITSCH, Media, PA (US); Joshua HARVEY, New York, NY (US)

(73) Assignees: New York University, New York, NY (US); Canaery Inc., Media, PA (US)

(21) Appl. No.: 18/577,660

(22) PCT Filed: Jul. 8, 2022
(86) PCT No.: PCT/US2022/036578

§ 371 Date: Jan. 8, 2024

Related U.S. Application Data
(60) Provisional application No. 63/220,361, filed on Jul. 9, 2021.

(51) Int. Cl. A01K 15/02

(52) U.S. Cl. CPC A01K 15/02

(57) ABSTRACT

An odor training and detection system can include multiple service animals. Each service animal can be provided with a means by which neural activity can be read from the olfactory system. Each service animal can be associated with an edge computing device containing an updateable local database and enabled with wireless communication. Each service animal can be equipped with one or multiple cloud-based servers and databases. A family of anchor odor sets and computational methods can enable the alignment of olfactory maps across individual animals into a common coordinate framework. Further disclosed is a means of computing and communicating (in a privacy-preserving manner if desirable) federated updates to olfactory decoding models between the local databases on the edge and the cloud database(s).

### CROSS-REFERENCE TO RELATED APPLICATIONS

This applications claims the benefit of and priority to U.S. Provisional Application No. 63/220,361, filed on Jul. 9, 2021, the contents of which are incorporated by reference in its entirety.

### TECHNICAL FIELD

The present disclosure relates generally to the field of scent, odor, and chemical detection. More particularly, the present disclosure relates to efficient federated training of multiple agents, which can include service animals equipped with a neural interface.

### BACKGROUND

Service animals can be used for scent detection and scent tracking across a variety of sectors and industries. Examples include police canine (K9) units used by border protection agencies across the globe, by drug enforcement agencies, or by search-and-rescue teams, Gambian rats trained to sniff out buried land mines, or truffle hunting pigs. The animals in these examples can have two things in common: (1) they remain in use in the 21^{st} century because their exquisite sense of smell is superior to and more versatile than any portable man-made chemical sensor developed to-date, and (2) they require lengthy and expensive individual training to report the presence of a small sub-set of highly specialized odors through behavioral report. Scent detection training currently constitutes a costly and laborious investment that is only good for the working life of an individual service animal.

### SUMMARY

Aspects and advantages of embodiments of the present disclosure will be set forth in part in the following description, or can be learned through practice of the embodiments.

The systems and methods of the present disclosure, in some embodiments, relate to an odor training and detection system. The odor training and detection system can include multiple service animals, each provided with a means by which neural activity can be read from the olfactory system, and each associated with an edge computing device containing an updateable local database and enabled with wireless communication. The odor training and detection system can include one or multiple cloud-based servers and databases. Further, the systems and methods of the present disclosure, in some embodiments, relate to a family of anchor odor sets and computational methods that enable the alignment of olfactory maps across individual animals into a common coordinate framework. Additionally, the systems and methods of the present disclosure, in some embodiments, relate to a means of computing and communicating (in a privacy-preserving manner if desirable) federated updates to olfactory decoding models between the local databases on the “edge” and the cloud database(s).

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

The details of one or more implementations of the subject matter described in this specification are set forth in the accompanying drawings and the description below. Other features, aspects, and advantages of the subject matter will become apparent from the description, the drawings, and the claims.

FIG. 1 illustrates a schematic of a scent detection system, according to an embodiment.

FIG. 2 illustrates a method of aligning neural signatures, according to an embodiment.

FIG. 3 illustrates a method of federated improvement of cloud-based scent decoding machine learning models, according to an embodiment.

Like reference numbers and designations in the various drawings indicate like elements.

### DETAILED DESCRIPTION

This disclosure relates generally to animal-based odor detection systems and methods for the federated training thereof. Federated training (e.g., federated learning, collaborative learning, etc.) can include a machine learning technique that trains an algorithm across multiple decentralized edge devices which hold local data without exchanging the data.

A neural interface to the olfactory system can be used to decode sniffed odorants from recorded neural activity. U.S. patent application Ser. No. 16/312,973 discusses a brain machine interface (e.g., bio-electronic nose), and is incorporated herein by reference. However, the neural signatures for the same odor may differ across individual animals, which may require each animal to be calibrated for every odor of interest, which can be lengthy, laborious, and costly.

Therefore, a need exists in the field for training methods which can enable the preservation of training experience beyond a service animal's working life. A further need exists for a method by which information about odors can be rapidly shared between individual animals. A further need exists to allow the periodic or one-time recalibration of an individual animal's neural responses if they change over the animal's lifetime. There is also a need for collecting such information without necessarily disclosing the location and time of this information source, which may be sensitive information.

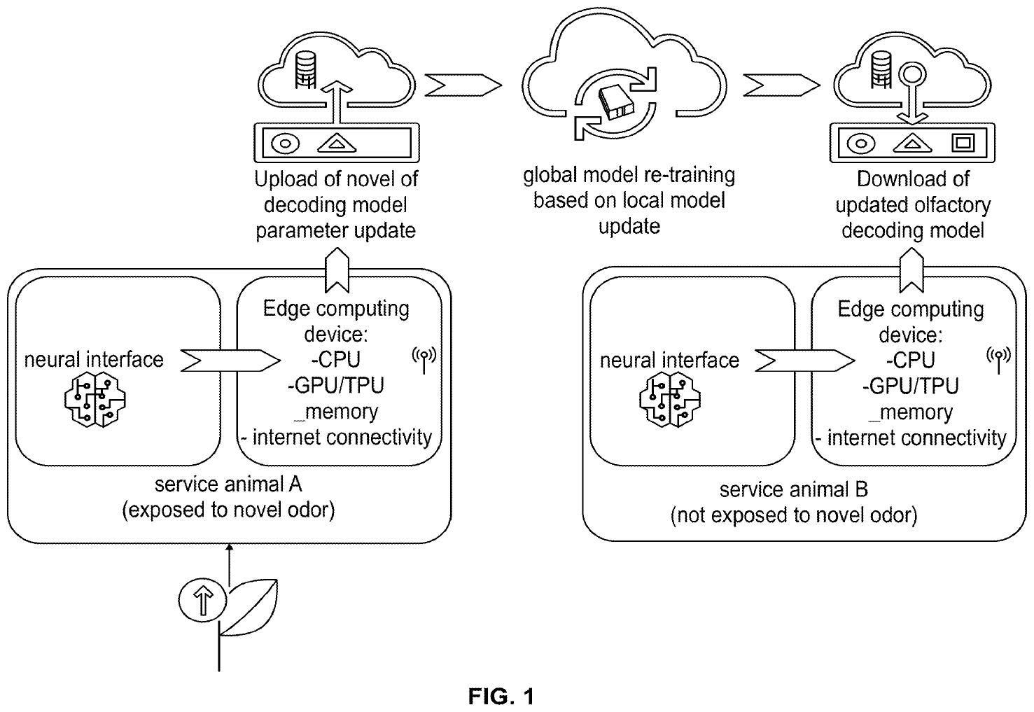

FIG. 1 illustrates a schematic of a scent detection system (e.g., operation scheme). One service animal, without loss of generality, service animal A, can be augmented with a neural interface and an edge computing device. Service animal A can be exposed to a novel (e.g., new) odor. The local edge device, which can be in possession of a local copy of the global decoding model, can convert the local representation of olfactory neural activity into the common coordinate framework and can compute an update of the model. The updated model can be encrypted. The updated model can be uploaded to the cloud server where it can be incorporated into the global model. The global model can be distributed to any number of augmented service animals on the network, represented in FIG. 1 without loss of generality by animal B.

A plurality of service animals (e.g., canines, pigs, ferrets, rats, mice, etc.) can be equipped with a means by which neural activity can be recorded from their respective olfactory systems. The service animals can have an exquisite (e.g., strong) sense of smell. In some embodiments, the means by which neural activity can be recorded from the olfactory systems of the service animals can include a microelectrode array implanted over the surface of the olfactory bulb. The electrode center-to-center distance can be optimized to enable optimal spatial sampling of glomerular activity, which, in some embodiments, can be achieved by matching the average radius of a glomerulus in the respective species. Electrode grids can be designed to spatially oversample glomeruli given their average size. Electrode center-to-center distance as well as electrode site size can vary across the spatial extent of a device to preferentially sample some portions of olfactory system over others. The service animals can be equipped with a system for the amplification, multiplexing, digitization, and/or wireless transmission (e.g., radio and antenna) of the neural signals. The service animals can be equipped with an edge computing device capable of demultiplexing and performing computations (e.g., tensor multiplication) on the neural data, and of communicating with a server in the cloud. The system can be a concatenation of commercially available components, or a single or multiple custom application-specific integrated circuit(s) (ASIC), or a combination of the above.

The method disclosed herein for the alignment of individual animal's olfactory neural signatures into a common coordinate framework (“CCF”) can use a panel of select anchor odors at specific concentrations (“anchors”). These anchors may not be unique, but they can include a family of odors with the following properties: (1) for each olfactory receptor there is at least one anchor in the anchor panel (e.g., anchor odor panel) that activates that receptor and (2) for each anchor, there is at least one type of olfactory receptor that is not activated by the anchor. The number of anchors can be less than or equal to the number of olfactory receptor types for the species (e.g., 500-1200) or the maximum number of simultaneously recorded glomeruli, whichever is smaller. In the simplest and most straightforward case, the anchors can be a set of odors at specific concentrations where each odor only activates a single glomerulus. In this case, the common coordinate framework can include the list of anchors (e.g., in an arbitrary but fixed order), and the transformation between the CCF and each animal can include the look-up table (e.g., a one-to-one (bijective) map) between the list of anchors and the centroid coordinates of maximal glomerular activation in response to the presentation of the respective anchor to the respective animal. This can be known as an anchor map. Olfactory receptors are conserved within and between species. Thus, different service animals can possess the same set of olfactory receptors in a slightly different spatial arrangement. Therefore, the anchor map can enable the identification of corresponding glomeruli across animals (e.g., within species, across species with a small loss in assignment accuracy, etc.). Thus, any olfactory neural activity pattern can be decomposed, from any animal, in the basis spanned by the anchors. To enable rapid transfer of odor exposure experience between animals, without loss of generality, one can assume that animal A is presented with a novel odor X, which elicits the (animal A-specific) neural activity pattern X_{A}. Using the anchor map M_{A} for animal A, that pattern can be first translated into the animal independent anchor representation X′=M_{A}(X_{A}). Then, using the inverse of the anchor map for animal B (M_{B}⁻¹), the animal B-specific neural activity pattern X_{B} can be predicted as X_{B}=M_{B}⁻¹(X′)=M_{B}⁻¹M_{A}(X_{A}). Even though animal B has not yet directly experienced odor X, following a procedure substantially similar to the one described above, it may be possible to detect its presence from animal B's olfactory neural activity.

While single-glomerular spatial resolution can be a sufficient condition for the method described above to function, it is not strictly necessary. The method can work even for embodiments where single-glomerular resolution is not achieved and if a smaller number of anchors is used than the number of interrogated glomeruli, to a degree that can be determined by inter-glomerular activity correlations over space and/or time. The number of interrogated individual glomeruli can set an upper bound for the spatial dimensionality of the signal and on the required number of anchor odors. Additional anchor odors beyond that upper bound may not be included, but may not significantly increase the method's performance.

The method disclosed herein for the federated improvement of cloud-based odor decoding can enable a central decoding model to be constantly updated and improved without revealing the presence or absence of any given odor at any given time or location to the central server. Since many of the compounds detectable by the enhanced service animals and their associated edge devices can be subject to privacy concerns or classification (e.g. diagnostic medical information, law enforcement operations, etc.), the odor decoding model stored in a central cloud database can be augmented by the individual experience of the edge devices without transmitting highly sensitive information. To achieve this, each edge device can first download the most recent version of the global model (e.g., global decoding model) in CCF. The central server and global model do not need to know the anchor map M, which can be restricted to the edge device. The local odor exposure experience can then be converted into the CCF locally on the edge device, and the model update (e.g., gradient) can be computed locally, encrypted, and transmitted to the cloud. In the cloud, the gradient can be used to update the global model, which can then in turn be downloaded to all active edge devices on the network.

A scent detection system for detecting volatile chemical compounds can include a plurality of animals (e.g., service animals) equipped with: a means of recording neural activity from an olfactory system or portion thereof, an edge computing device equipped with a local database and wireless communication, and one or multiple server-side databases in a cloud.

In some embodiments, the means of recording neural activity includes a neural interface. The neural interface can include a communication pathway between electrical activity of a brain and an external device (e.g., computer). In some embodiments, the plurality of animals can be configured to access a global decoding model on the cloud.

In some embodiments, the edge computing device is configured to convert a local representation of olfactory neural activity into a common coordinate framework. The edge computing device can include a local copy of a global decoding model. The edge computing device can be configured to compute an update of the local copy of the global decoding model. The edge computing device can be configured to upload the updated model to the cloud. The edge computing device can be configured to incorporate the updated model into the global decoding model on the cloud.

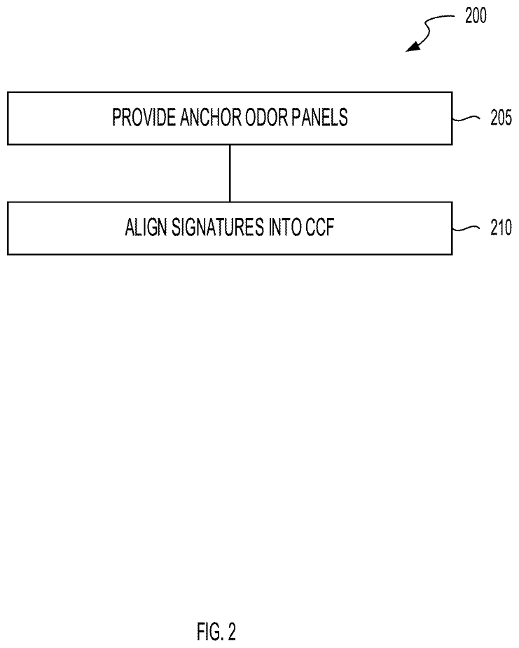

FIG. 2 illustrates a method 200 of aligning neural signatures. The method 200 can include providing a family of anchor odor panels (BLOCK 205). The family of anchor odor panels can include a plurality of anchors. The method 200 can include aligning individual animal's olfactory neural signatures into a common coordinate framework (“CCF”) (BLOCK 210). The common coordinate framework can include a list of anchors.

The method 200 can include identifying an odor and a first corresponding glomerulus of the odor for a first animal of a species. The method 200 can include identifying the odor and a second corresponding glomerulus of the odor for a second animal of the species. The method 200 can include identifying an odor and a first corresponding glomerulus of the odor for a first animal of a first species. The method 200 can include identifying the odor and a second corresponding glomerulus of the odor for a second animal of a second species. The second species can be different from the first species.

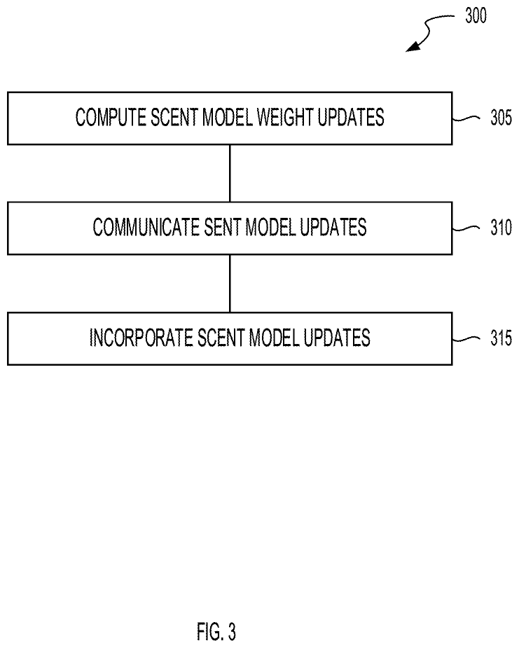

FIG. 3 illustrates a method 300 of federated improvement of cloud-based scent decoding machine learning models. The method 300 can include computing scent model weight updates (BLOCK 305). For example, the method 300 can include computing scent model weight updates on an edge device in CCF coordinates. A common coordinate framework can include the common coordinate framework coordinates. The common coordinate framework can include a list of anchors. The method 300 can include communicating encrypted scent model updates (BLOCK 310). For example, the method 300 can include communicating encrypted scent model updates to a cloud. The method 300 can include incorporating scent model updates (BLOCK 315). For example, the method 300 can include incorporating scent model updates into cloud-based model in cryptographic space.

The method 300 can include acquiring a global model. The method 300 can include identifying an odor and a first corresponding glomerulus of the odor for a first animal of a species. The method 300 can include identifying the odor and a second corresponding glomerulus of the odor for a second animal of the species. The method 300 can include identifying an odor and a first corresponding glomerulus of the odor for a first animal of a first species. The method 300 can include identifying the odor and a second corresponding glomerulus of the odor for a second animal of a second species. The second species can be different from the first species.

The subject matter and the operations described in this specification can be implemented in digital electronic circuitry, or in computer software, firmware, or hardware, including the structures disclosed in this specification and their structural equivalents, or in combinations of one or more of them. The subject matter described in this specification can be implemented as one or more computer programs, e.g., one or more circuits of computer program instructions, encoded on one or more computer storage media for execution by, or to control the operation of, data processing apparatus. Alternatively or in addition, the program instructions can be encoded on an artificially generated propagated signal, e.g., a machine-generated electrical, optical, or electromagnetic signal that can be generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium may not be a propagated signal, a computer storage medium can be a source or destination of computer program instructions encoded in an artificially generated propagated signal. The computer storage medium can also be, or be included in, one or more separate components or media (e.g., multiple CDs, disks, or other storage devices).

The operations described in this specification can be performed by a data processing apparatus on data stored on one or more computer-readable storage devices or received from other sources. The term “data processing apparatus” or “computing device” encompasses various apparatuses, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a circuit, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more circuits, subprograms, or portions of code). A computer program can be deployed to be executed on one computer or on multiple computers that are located at one site or distributed across multiple sites and interconnected by a communication network.

Processors suitable for the execution of a computer program include, by way of example, microprocessors, and any one or more processors of a digital computer. A processor can receive instructions and data from a read only memory or a random access memory or both. The elements of a computer are a processor for performing actions in accordance with instructions and one or more memory devices for storing instructions and data. A computer can include, or be operatively coupled to receive data from or transfer data to, or both, one or more mass storage devices for storing data, e.g., magnetic, magneto optical disks, or optical disks. A computer need not have such devices. Moreover, a computer can be embedded in another device, e.g., a personal digital assistant (PDA), a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The implementations described herein can be implemented in any of numerous ways including, for example, using hardware, software or a combination thereof. When implemented in software, the software code can be executed on any suitable processor or collection of processors, whether provided in a single computer or distributed among multiple computers.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

A computer employed to implement at least a portion of the functionality described herein may comprise a memory, one or more processing units (also referred to herein simply as “processors”), one or more communication interfaces, one or more display units, and one or more user input devices. The memory may comprise any computer-readable media, and may store computer instructions (also referred to herein as “processor-executable instructions”) for implementing the various functionalities described herein. The processing unit(s) may be used to execute the instructions. The communication interface(s) may be coupled to a wired or wireless network, bus, or other communication means and may therefore allow the computer to transmit communications to or receive communications from other devices. The display unit(s) may be provided, for example, to allow a user to view various information in connection with execution of the instructions. The user input device(s) may be provided, for example, to allow the user to make manual adjustments, make selections, enter data or various other information, or interact in any of a variety of manners with the processor during execution of the instructions.

The various methods or processes outlined herein may be coded as software that is executable on one or more processors that employ any one of a variety of operating systems or platforms. Additionally, such software may be written using any of a number of suitable programming languages or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement features of the solution discussed above. The computer readable medium or media can be transportable, such that the program or programs stored thereon can be loaded onto one or more different computers or other processors to implement various aspects of the present solution as discussed above.

The terms “program” or “software” are used herein to refer to any type of computer code or set of computer-executable instructions that can be employed to program a computer or other processor to implement various aspects as discussed above. One or more computer programs that when executed perform methods of the present solution need not reside on a single computer or processor, but may be distributed in a modular fashion amongst a number of different computers or processors to implement various aspects of the present solution.

Computer-executable instructions may be in many forms, such as program modules, executed by one or more computers or other devices. Program modules can include routines, programs, objects, components, data structures, or other components that perform particular tasks or implement particular abstract data types. The functionality of the program modules can be combined or distributed as desired in various implementations.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

As used herein, the singular forms “a”, “an” and “the” include plural referents unless the context clearly dictates otherwise. Thus, for example, the term “a member” is intended to mean a single member or a combination of members, “a material” is intended to mean one or more materials, or a combination thereof.

As used herein, the terms “about” and “approximately” generally mean plus or minus 10% of the stated value. For example, about 0.5 would include 0.45 and 0.55, about 10 would include 9 to 11, about 1000 would include 900 to 1100.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

Any references to implementations or elements or acts of the systems and methods herein referred to in the singular can include implementations including a plurality of these elements, and any references in plural to any implementation or element or act herein can include implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation, and references to “an implementation,” “some implementations,” “an alternate implementation,” “various implementations,” “one implementation” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Elements other than ‘A’ and ‘B’ can also be included.

The systems and methods described herein may be embodied in other specific forms without departing from the characteristics thereof. The foregoing implementations are illustrative rather than limiting of the described systems and methods.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

What is claimed is:

1. A scent detection system for detecting volatile chemical compounds comprising:
a plurality of animals equipped with:
a means of recording neural activity from an olfactory system or portion thereof;
an edge computing device equipped with a local database and wireless communication; and
one or multiple server-side databases in a cloud.

2. The system of claim 1, wherein the means of recording neural activity comprises a neural interface.

3. The system of claim 1, wherein the edge computing device is configured to convert a local representation of olfactory neural activity into a common coordinate framework.

4. The system of claim 1, wherein the edge computing device comprises a local copy of a global decoding model.

5. The system of claim 1, wherein the edge computing device comprises a local copy of a global decoding model and the edge computing device is configured to compute an update of the local copy of the global decoding model.

6. The system of claim 1, wherein the edge computing device comprises a local copy of a global decoding model and the edge computing device is configured to:
compute an update of the local copy of the global decoding model; and
upload the updated model to the cloud.

7. The system of claim 1, wherein the edge computing device comprises a local copy of a global decoding model and the edge computing device is configured to:
compute an update of the global decoding model;
upload the updated model to the cloud; and
incorporate the updated model into the global decoding model on the cloud.

8. The system of claim 1, wherein the plurality of animals are configured to access a global decoding model on the cloud.

9. A method, comprising:
providing a family of anchor odor panels; and
aligning individual animal's olfactory neural signatures into a common coordinate framework.

10. The method of claim 9, wherein the family of anchor odor panels comprises a plurality of anchors.

11. The method of claim 9, wherein the common coordinate framework comprises a list of anchors.

12. The method of claim 9, further comprising:
identifying an odor and a first corresponding glomerulus of the odor for a first animal of a species; and
identifying the odor and a second corresponding glomerulus of the odor for a second animal of the species.

13. The method of claim 9, further comprising:
identifying an odor and a first corresponding glomerulus of the odor for a first animal of a first species; and
identifying the odor and a second corresponding glomerulus of the odor for a second animal of a second species, the second species different from the first species.

14. The method of claim 9, further comprising decomposing an olfactory neural activity pattern in a basis spanned by a plurality of anchors.

15. A method of federated improvement of cloud-based scent decoding machine learning models, the method comprising:
computing scent model weight updates on an edge device in common coordinate framework coordinates;
communicating encrypted scent model updates to a cloud; and
incorporating scent model updates into cloud-based model in cryptographic space.

16. The method of claim 15, further comprising acquiring a global model.

17. The method of claim 15, wherein a common coordinate framework comprises the common coordinate framework coordinates.

18. The method of claim 15, wherein:
a common coordinate framework comprises the common coordinate framework coordinates; and
the common coordinate framework comprises a list of anchors.

19. The method of claim 15, further comprising:
identifying an odor and a first corresponding glomerulus of the odor for a first animal of a species; and
identifying the odor and a second corresponding glomerulus of the odor for a second animal of the species.

20. The method of claim 15, further comprising:
identifying an odor and a first corresponding glomerulus of the odor for a first animal of a first species; and
identifying the odor and a second corresponding glomerulus of the odor for a second animal of a second species, the second species different from the first species.
